(19) United States
(12) Patent Application Publication
(10) Pub. No.: US 2023/0043897 A1
(43) Pub. Date: Feb. 9, 2023

(54) Personal Care Capsule Manufacture and Storage

(71) Applicant: ELC MANAGEMENT LLC, Melville, NY (US)

(72) Inventors: Matthew J. Hong, Dix Hills, NY (US); Jill Marie Villacci, Merrick, NY (US); Rita Wei-Yu Chen, New York, NY (US); Marc Emile Lechanoine, New York, NY (US); Timothy Hugh Calvert, Brentwood, TN (US); Gianluca Mattaroccia, Sunnyside, NY (US)

(21) Appl. No.: 17/444,554

(22) Filed: Aug. 5, 2021

(51) Int. Cl. A45D 40/00; B65D 83/04

(52) U.S. Cl. CPC A45D 40/00; B65D 83/0409

(57) ABSTRACT

A personal care capsule includes a capsule shell and a personal care fluid contained within. The personal care capsule is manufactured according to a method involving freezing a membrane solution within a capsule shell mold and rinsing the capsule shell in sodium alginate. A storage device for a plurality of personal care capsules includes a container having an access portal, a capsule rack including a plurality of capsule receptacles, and an access portal cap adapted to cover the access portal. The capsule rack is adapted to move through the access portal between a storage position and a use position.

### FIELD OF THE DISCLOSURE

The present disclosure relates body care products, and specifically relates to personal care capsules, a method of manufacturing personal care capsules, and a storage device for personal care capsules.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Delivery and storage solutions for personal care products, such as skin or hair care products, must take into account a number of design considerations. Personal care products need to be stored in a manner that maintains their potency. For example, many skin care products degrade when exposed to air and/or other unsealed environments due to oxidation. Heat and UV radiation can also cut short the shelf life of a personal care product. Personal care products are optimally stored in a sanitary manner that does not allow bacteria to contaminate the product. For example, optimally, a user is able to apply a personal care product without bacteria from the user's hand contaminating future applications of the product. At the same time, personal care product storage should allow and facilitate access to the personal care product. Personal care storage devices ideally also reduce harm to the environment by being recyclable or biodegradable.

Accordingly, there is a need for improved personal care storage devices having improved functionalities.

### SUMMARY

The disclosed personal care capsules and storage devices optimally balance the competing design considerations discussed above. The disclosed personal care capsules are able to contain, maintain, and deliver high-potency concentrates. The storage devices are able to limit the personal care capsules' exposure to degrading forces while simultaneously allowing easy access to the personal care capsules. The personal care capsules are biodegradable, and the storage devices are recyclable, and are thus environmentally friendly. In addition to being functional, the storage devices and the disclosed personal care capsules are aesthetically pleasing.

In accordance with an example, a storage device for a plurality of personal care capsules includes a container, a capsule rack, and an access portal cap. The container includes a base, an enclosed volume, and an access portal. The capsule rack includes a plurality of capsule receptacles. The capsule rack is adapted to move through the access portal between a storage position in which each of the plurality of capsule receptacles are located within the enclosed volume and a use position in which at least one of the plurality of capsule receptacles is located outside the enclosed volume. The access portal cap is adapted to cover the access portal when the capsule rack is in the storage position.

In some forms, the storage device may include a securement mechanism adapted to secure the access portal cap to the container when the capsule rack is in the storage position. Each capsule receptacle may include a support member, and the capsule rack may include a spine operably coupled with each of the capsule receptacles at the support member. The support member may include at least one perforation to allow drainage as the capsule rack moves from the storage position to the use position.

In some forms, the capsule rack may include a spine connected to each of the capsule receptacles, the capsule rack may include a plurality of receptacle heights, and a single capsule receptacle may be provided at each receptacle height. The capsule receptacles may be substantially aligned to form a single column.

In further forms, the container may include a plurality of access portals and a plurality of enclosed volumes, each of the plurality of access portals being associated with a respective one of the plurality of enclosed volumes. The container may further include a plurality of capsule racks including capsule receptacles, each capsule rack associated with a respective one of the plurality of access portals, each capsule rack adapted to move through the respective one of the plurality of access portals between a storage position with all of the capsule receptacles of the capsule rack located within the respective one of the plurality of enclosed volumes and a use position with at least one of the capsule receptacles of the capsule rack located outside the respective one of the plurality of enclosed volumes. Each of the plurality of access portals may be located at the same height and arranged linearly.

In other forms, the plurality of access portals may include a first subset and a second subset, the first subset located at a first height, and the second subset located at a second height that is greater than the first height. In still other forms, the plurality of capsule racks may include a first subgroup having capsule receptacles adapted to accommodate personal care capsules of a first size and a second subgroup having capsule receptacles adapted to accommodate personal care capsules of a second size.

In some forms, the storage device may include a base adapted for selective connection to the container. The base may include a stand adapted for placement on a flat surface, and the stand may be connected to a holder adapted to selectively hold the container.

In accordance with an example, a method of manufacturing a personal care capsule includes preparing a membrane solution including 0.5-6% calcium lactate, pouring the membrane solution into a capsule shell mold, and freezing the personal care solution within the capsule shell mold. The method further includes removing the personal care solution from the capsule mold as a capsule shell, bathing the capsule shell in 1.0-0.5% sodium alginate to create the personal care capsule, and rinsing the personal care capsule in water.

In some forms, the method may include freezing the personal care capsule to prepare for loading into to storage device and placing the frozen personal care capsule on a capsule rack of a storage device. The method may include submerging the personal care capsule in a storage solution located within a container of the storage device. The personal care fluid may include an active ingredient and the storage solution may include the same active ingredient.

In accordance with an example, a personal care capsule includes a capsule shell including 0.5-6% calcium lactate, the capsule shell having an interior volume capable of containing a mass of a personal care fluid between 0.5 grams and 5 grams. A personal care fluid is contained within the capsule shell, the personal care fluid including an active ingredient.

In some forms, the capsule shell may include an indicator indicating the active ingredient of the personal care fluid contained within the capsule shell. The capsule shell may be biodegradable. The personal care capsule shell may have a diameter between 10 and 15 mm.

### BRIEF DESCRIPTION OF THE DRAWINGS

The figures described herein depict various aspects of the system and methods disclosed herein. It should be understood that each figure depicts an example of aspects of the present systems and methods.

FIG. 1 is a front view of a first example storage device for a plurality of personal care capsules in a storage position, the first example storage device including a base, the personal care capsules arranged in a single column in accordance with various embodiments.

FIG. 2 is a front view of the first example storage device of FIG. 1 in a use position in accordance with various embodiments.

FIG. 3 is a front view of a second example storage device including three capsule racks and a hanging frame, an access portal for each capsule rack located at the same height, in accordance with various embodiments.

FIG. 4 is a front view of a third example storage device including a plurality of capsule racks secured to a hanging frame, an access portal for each capsule rack located at a different height, in accordance with various embodiments.

FIG. 5 is a front view of a fourth example storage device manufactured using additive manufacturing.

FIG. 6 is a cut-away view of a personal care capsule in accordance with various embodiments.

FIG. 7 shows a capsule shell mold being filled with a membrane solution in accordance with various embodiments.

FIG. 8 illustrates schematically a method of manufacturing a personal care capsule in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various examples. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

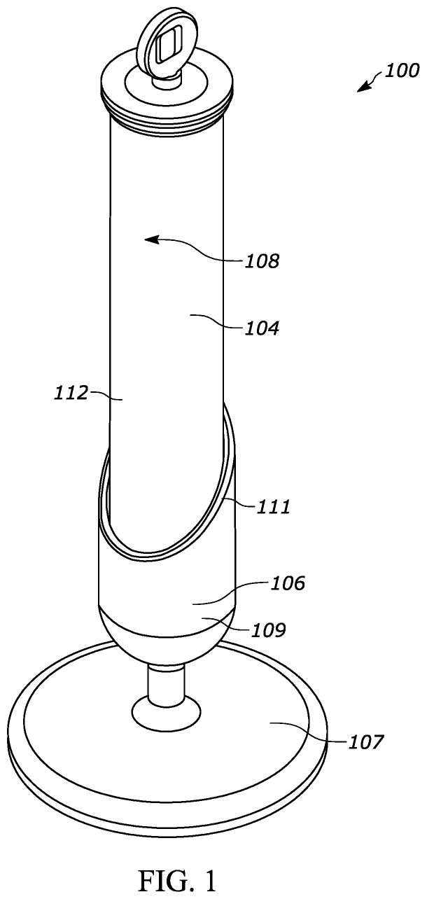

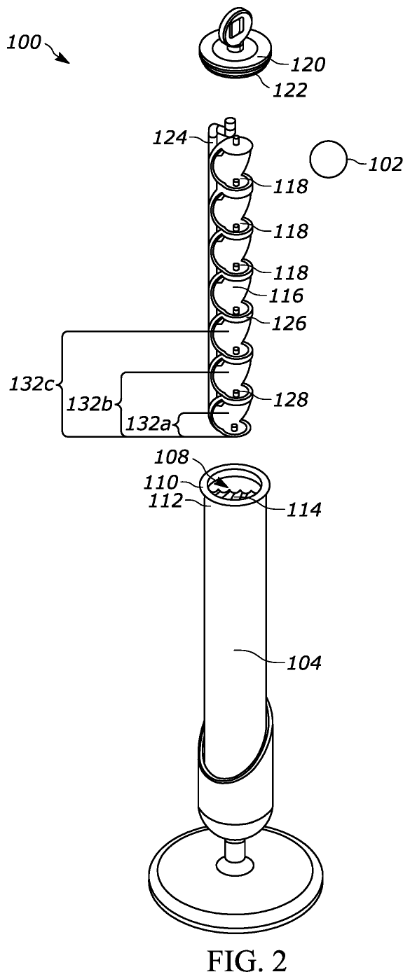

Turning to the figures, FIG. 1 shows a storage device 100 for a plurality of personal care capsules 102 (shown in FIG. 2). The storage device 100 has a container 104 that is supported by a base 106. The base 106 is located below the container 104 and is adapted to be set on top of a flat surface, such as a vanity top. The container 104 is operably connected with the base 106 and is centered over the standing base 106 such that the container 104 is securely balanced in an upright position by the base 106. The base 106 shown in FIG. 1 allows selective connection to the container 104 such that the container 104 can be removed from the base 106 if, for example, a user wishes to hold the container 104 while retrieving a personal care capsule 102. The base 106 includes a stand 107 that may be adapted for placement on a flat surface, and the stand is connected to a holder 109 adapted to selectively hold the container 104. The holder 109 shown in FIG. 1 is cup-shaped and may optionally include an asymmetric top edge 111. In other arrangements, the holder 109 may have a symmetric top edge 111 or a different shape that partially or fully surrounds the container 104.

The container 104 has an enclosed volume 108 that is accessible via an access portal 110. In the storage device 100 shown in FIG. 1, the enclosed volume 108 is formed by an outer wall 112 that is generally cylindrical. In other arrangements not depicted herein, the enclosed volume 108 may be formed by an outer wall 112 that has any of a variety of other three-dimensional shapes, such as a cuboid, a triangular prism, a pentagonal prism, or the like. The outer wall 112 shown in FIG. 1 may be formed from glass or plastic, such as an acrylic. The outer wall 112 may be translucent or light-transmissive in order to allow the personal care capsules to be visible through the outer wall 112. In some approaches, the outer wall 112 may be UV-filtering in order to prevent UV radiation from reducing the shelf life of the personal care capsules 102. For purposes of this application, “personal care” is descriptive of products that include at least skin care products, hair care products, and makeup and color products. For purposes of this application, a “fluid” or “solution” may be a fluid, a liquid, or a non-flowing gel or gelatinous substance.

With particular reference to FIG. 2, a storage solution 114 may be stored within the enclosed volume 108. The access portal 110 in FIGS. 1 and 2 is located at the top of the container 104. In other arrangements not depicted herein, the access portal 110 may be located on a side or on a bottom of the container 104. If the storage device 100 is to contain a storage solution 114, the access portal 110 may be positioned in a manner that allows the personal care capsules 102 to be removed from the container 104 while retaining (i.e., without spilling, leaking, or otherwise emitting) the storage solution 114. For example, if the access portal 110 is located on a side of the container 104, the outer wall 112 may include a lip or other retention mechanism to prevent spillage.

As shown in FIG. 1, a capsule rack 116 is positioned within the container 104 and includes a plurality of capsule receptacles 118. In the storage position depicted in FIG. 1, each of the plurality of capsule receptacles 118 are located within the enclosed volume 108. The capsule rack 116 is adapted to move through the access portal 110 from the storage position shown in FIG. 1 to the use position shown in FIG. 2. In the use position, at least one of the plurality of capsule receptacles 118 is located outside the enclosed volume 108. In particular, in the use position, a user is able to remove a personal care capsule 102 from a capsule receptacle 118. After use, the user may return the capsule rack 116 to the storage position within the container 104.

When the storage device 100 is in the storage position shown in FIG. 1, an access portal cap 120 is adapted to cover the access portal 110. A securement mechanism 122 may secure the access portal cap 120 to the container 104. The securement mechanism 122 prevents spills should the storage device 100 be tipped over or for purposes of travel. For example, in FIG. 1, the access portal cap 120 has a friction fit with the outer wall 112 of the container 104 that serves as the securement mechanism 122. Alternately, the access portal cap 120 may be screwed into the outer wall 112 via a threaded connection or may be secured to the outer wall 112 by a separate engaging mechanism such as, for example, a lock or clasp. The securement mechanism 122 may be childproof so that access to the personal care capsules 102 is limited to adults capable of opening the securement mechanism 122.

As shown in FIG. 2, the capsule rack 116 includes a spine 124 to which each of the capsule receptacles 118 are operably coupled. The spine 124 may be flexible in order to allow the capsule rack 116 to flex and bend as needed when moving through the access portal 110 and may extend in an axial or longitudinal direction. Each capsule receptacle 118 includes a support member 126 configured to hold a personal care capsule 102. The capsule rack 116 moves through the storage solution 114 contained within the enclosed volume 108 when moving from the storage position to the use position or vice versa. The support member 126 may include at least one perforation or throughbore 128 to allow drainage and to facilitate this movement. The at least one perforation 128 may be a single aperture or may be a plurality of apertures. Particularly when one or more of the capsule receptacles 118 are empty (perhaps as a result of a personal care capsule previously having been used), the perforation or perforations 128 help to prevent the support member 126 from retaining storage solution 114 and subsequently spilling the storage solution 114 when a user retrieves another personal care capsule.

As shown in FIG. 2, the capsule rack 116 includes a plurality of receptacle heights 132 (132a, 132b, 132c . . . ), meaning places where one or more of the capsule receptacles 118 are adapted to hold a personal care capsule 102. The receptacle heights 132 may be measured from a top or a bottom of the spine 124. In the arrangement shown in FIG. 2, a single capsule receptacle 118 is provided at each receptacle height 132. As a result, the capsule receptacles 118 are substantially aligned to form a single column. Put another way, each capsule receptacle 118 extends from the spine 124 at an identical radial location but a different receptacle height 132 (i.e., a different longitudinal or axial position along the spine 124).

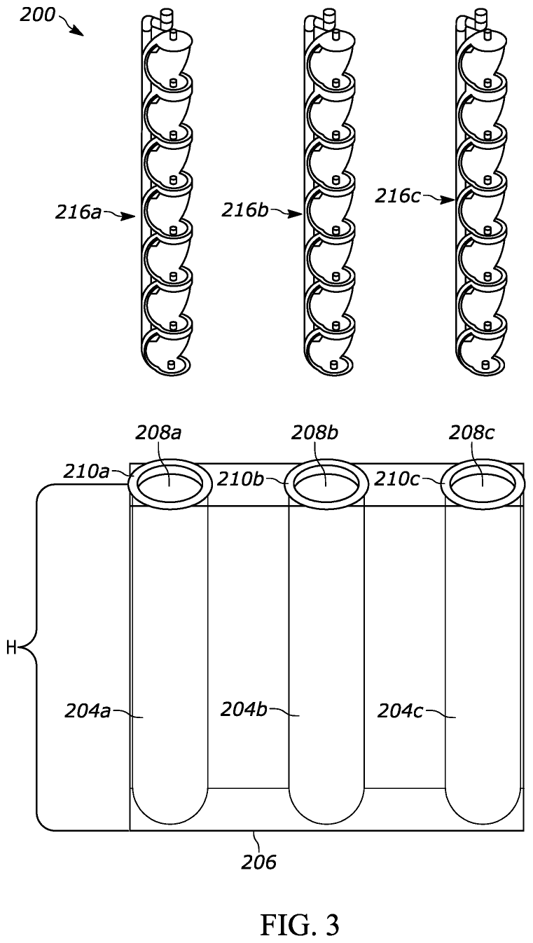

FIG. 3 illustrates an alternative storage device 200 with features similar to those discussed with respect storage device 100. It will be appreciated that the storage device 200 illustrated in FIG. 3 may include similar features to the storage device 100, and thereby elements illustrated in FIG. 3 are designated by similar reference numbers indicated on the embodiment illustrated in FIGS. 1-3, increased by 100. Accordingly, these features will not be described in substantial detail. Further, it is appreciated that any combination or sub-combination of features described in regard to the storage device 200 may be incorporated into the storage device 100, and vice-versa. Instead of a base 106 configured to support a single capsule rack 116, storage device 200 has a base 206 configured to support multiple capsule racks 216 (216a, 216b, 216c). In the arrangement shown in FIG. 3, three containers 204a, 204b, and 204c extend through and are secured in the base 206. As a result, the storage device 200 includes a plurality of access portals 210 (210a, 210b, and 210c) and a plurality of enclosed volumes 208 (208a, 208b, 208c), each of the plurality of access portals 210 associated with a respective one of the plurality of enclosed volumes 208. Each capsule rack 216 (216a, 216b, and 216c) is adapted to move through the respective one of the plurality of access portals 410 between a storage position and a use position. In the arrangement shown in FIG. 3, each of the plurality of access portals 210 (210a, 210b, and 210c) are located at the same height H and are arranged linearly.

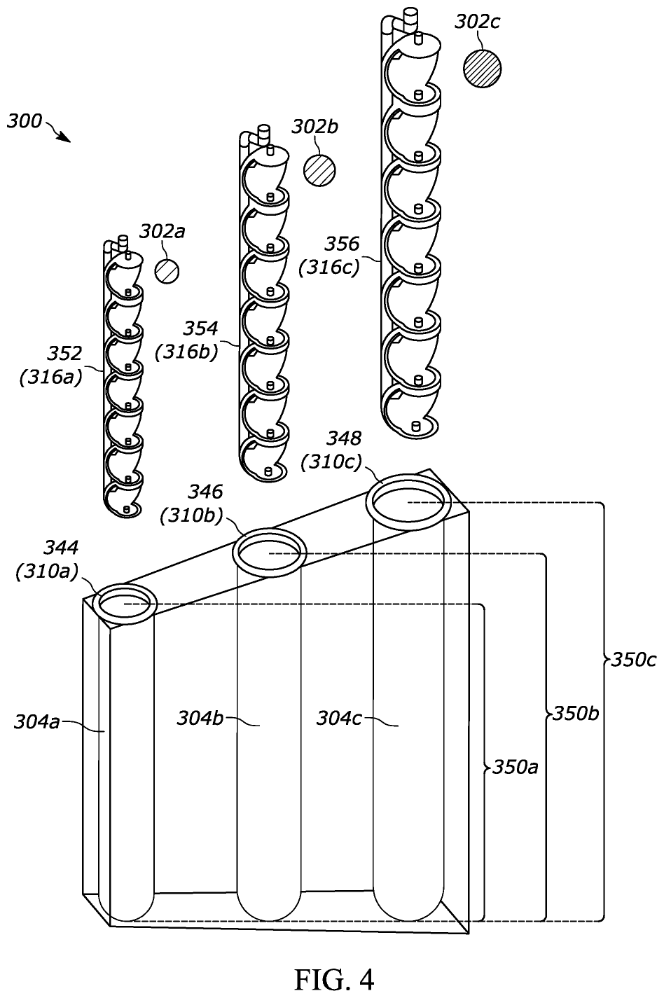

FIG. 4 illustrates an alternative storage device 300 with features similar to those discussed with respect storage devices 100 and 200. It will be appreciated that the storage device 300 illustrated in FIG. 4 may include similar features to the storage devices 100 and 200, and thereby elements illustrated in FIG. 4 are designated by similar reference numbers indicated on the embodiment illustrated in FIGS. 1-3, increased by 100 or 200. Accordingly, these features will not be described in substantial detail. Further, it is appreciated that any combination or sub-combination of features described in regard to the storage device 300 may be incorporated into the storage devices 100 and 200, and vice-versa. In the arrangement shown in FIG. 4, the plurality of containers 304 (304a, 304b, 304c) have a stepped arrangement. In particular, the plurality of access portals 310 of the plurality of containers 304 include a first subset 344 of access portals 310 (here, 310a), a second subset 346 of access portals 310 (here, 310b), and a third subset 348 of access portals 310 (here, 310c). While FIG. 4 includes only a single access portal 310 in each of subsets 344, 346, and 348, more than one access portal 310 may be included in each subset. The first subset 344 is located at a first subset height 350a, the second subset 346 is located at a second subset height 350b, and the third subset 348 is located at a third height 350c. In the arrangement shown in FIG. 4, the first subset height 350a is less than the second subset height 350b, and the second subset height 350b is less than the third subset height 350c. However, in other arrangements the relative subset heights may exhibit other patterns for functionality, manufacturability, and aesthetics. In some arrangements, the first subset, second subset, and third subset are associated with personal care capsules containing different active ingredients.

In the arrangement shown in FIG. 4, the plurality of capsule racks 316 of the plurality of containers 304 are adapted to accommodate personal care capsules 302 of different sizes. The plurality of capsule racks 316 include a first subgroup 352 associated with personal care capsules 302a of a first size, a second subgroup 354 associated with personal care capsules 302b of a second size, and a third subgroup 356 associated with personal care capsules 302c of a third size. While FIG. 4 includes only a single access portal 310 in each of subgroups 3352, 354, and 356, more than one access portal 310 may be included in each subgroup. In FIG. 4, the first subgroup 352, second subgroup 354, and third subgroup 356 are associated with the first subset 344, second subset 346, and third subset 348 respectively. However, in other arrangements not shown herein, the height of the access portals 310 may not correspond with the size of personal care capsule 302 that the respective capsule racks 316 are designed to accommodate.

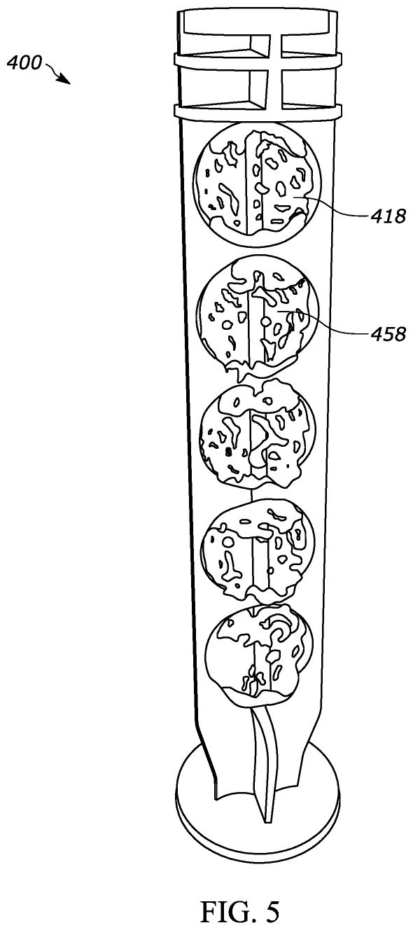

FIG. 5 illustrates an alternative storage device 400 with features similar to those discussed with respect storage devices 100, 200, and 300. It will be appreciated that the storage device 400 illustrated in FIG. 5 may include similar features to the storage devices 100, 200, and 300 and thereby elements illustrated in FIG. 5 are designated by similar reference numbers indicated on the embodiment illustrated in FIGS. 1-4, increased by 100, 200, or 300. Accordingly, these features will not be described in substantial detail. Further, it is appreciated that any combination or sub-combination of features described in regard to the storage device 300 may be incorporated into the storage devices 100, 200, and 300 and vice-versa. The storage device 400 in FIG. 5 may be manufactured partially or completely using additive manufacturing, such as 3D printing. The storage device 400 may be digitally defined by a computer-aided design (CAD) software that creates a file for manufacture of the storage device 400. The file guides the path of a nozzle or print head as it deposits layers of material to form the storage device 400, or a laser or electron beam selectively melts material that fuses to form the storage device 400. The shape of the storage device 400 may minimize the amount of material necessary to create functional storage device 400 for the sake of reducing the cost of manufacture. For example, in the arrangement shown in FIG. 4, the capsule receptacles 418 each have a web-like surface 458 that reduces the amount of material needed to make each capsule receptacle 418.

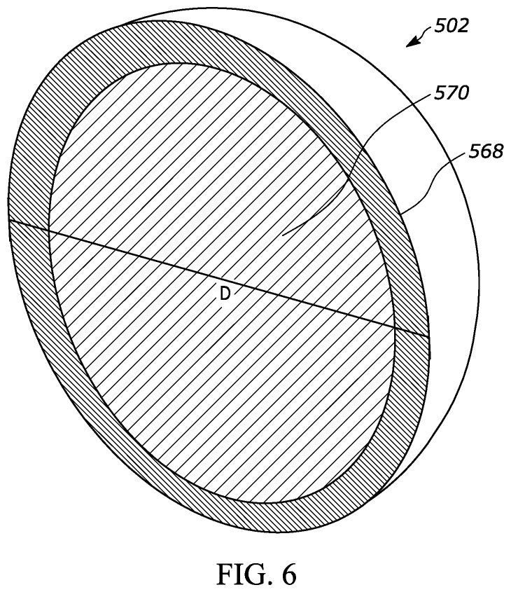

FIG. 6 illustrates an example personal care capsule 502 which may include similar features as any of the personal care capsules 102, 202, 302, or vice versa. The personal care capsule 502 has a capsule shell 568 and a personal care fluid 570 contained within the capsule shell 568. The capsule shell 568 includes 0.5-6% calcium lactate and is biodegradable. The capsule shell 568 has an interior volume capable of containing a mass of personal care fluid 570 between about 0.5 grams and 5 grams. An outer diameter D of the capsule shell 568 is between about 10 and 15 mm. The capsule shell 568 may include an indicator indicating the active ingredient of the personal care fluid 570 contained within the capsule shell 568. For example, the capsule shell may include or be dyed a certain color that, according to a set of instructions, indicates a particular active ingredient. The size of the personal care capsule 502 may be optimized to deliver an appropriate single dose of the active ingredient.

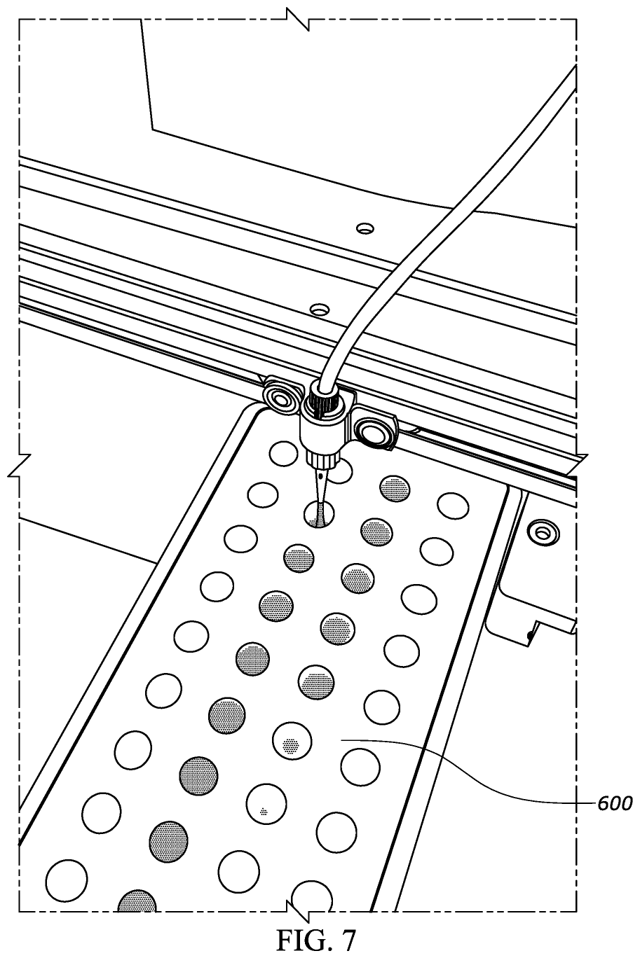

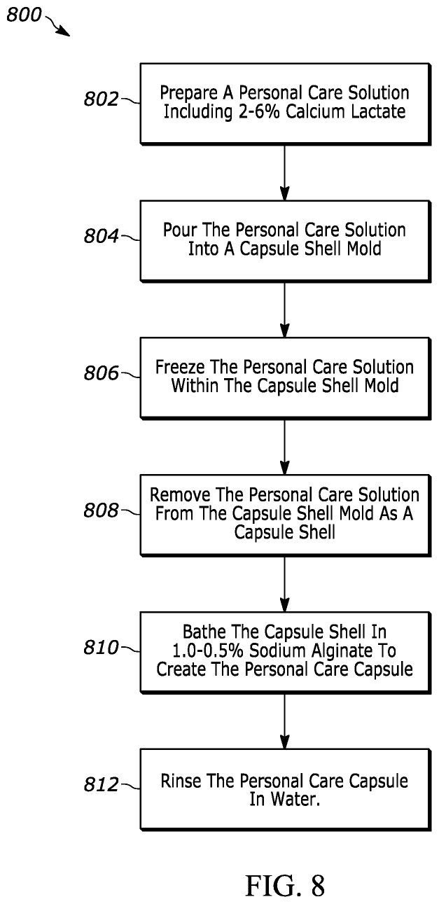

The process of manufacturing a personal care capsule 502 is shown and discussed in FIGS. 7 and 8. FIG. 7 illustrates a capsule shell mold 600 in which a capsule shell 568 is formed. As shown schematically in FIG. 8, a method 800 of manufacturing a personal care capsule 502 includes preparing a membrane solution including 0.5-6% calcium lactate, shown at a step 802. At a step 804, the membrane solution is poured into a capsule shell mold such as capsule shell mold 600. At a step 806, the membrane solution is frozen within the capsule shell mold. At a step 808, the membrane solution is removed from the capsule mold as a capsule shell, such as capsule shell 568. At a step 810, the capsule shell is bathed in 1.0-0.5% sodium alginate to create the personal care capsule 502. At a step 812, the personal care capsule 502 is rinsed in water.

The method 800 may further include placing the personal care capsule 602 on a capsule rack (such as capsule rack 116). The method 800 may further include submerging the personal care capsule 502 in a storage solution (such as storage solution 114) located within a container (such as container 104) of the storage device. The personal care fluid 570 may include an active ingredient and the storage solution may include the same active ingredient. This may prevent osmosis from leaching the active ingredient from the personal care capsule 502 prior to application.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive, and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The claimed invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

The patent claims at the end of this patent application are not intended to be construed under 35 U.S.C. § 112(f) unless traditional means-plus-function language is expressly recited, such as “means for” or “step for” language being explicitly recited in the claim(s).

What is claimed is:

1. A storage device for a plurality of personal care capsules, the storage device comprising:
a container including a base, an enclosed volume, and an access portal; and
a capsule rack including a plurality of capsule receptacles, the capsule rack adapted to move through the access portal between a storage position in which each of the plurality of capsule receptacles are located within the enclosed volume and a use position in which at least one of the plurality of capsule receptacles is located outside the enclosed volume.

2. The storage device of claim 1, further comprising an access portal cap adapted to cover the access portal when the capsule rack is in the storage position.

3. The storage device of claim 2, further comprising a securement mechanism adapted to secure the access portal cap to the container when the capsule rack is in the storage position.

4. The storage device of claim 1, wherein each capsule receptacle includes a support member, and the capsule rack including a spine operably coupled with each of the capsule receptacles at the support member.

5. The storage device of claim 4, wherein the support member includes at least one perforation to allow drainage as the capsule rack moves from the storage position to the use position.

6. The storage device of claim 1, the capsule rack including a spine connected to each of the capsule receptacles, the capsule rack including a plurality of receptacle heights, a single capsule receptacle provided at each receptacle height, wherein the capsule receptacles are substantially aligned to form a single column.

7. The storage device of claim 1, wherein the container further includes a plurality of access portals and a plurality of enclosed volumes, each of the plurality of access portals being associated with a respective one of the plurality of enclosed volumes, and further comprising a plurality of capsule racks including capsule receptacles, each capsule rack associated with a respective one of the plurality of access portals, each capsule rack adapted to move through the respective one of the plurality of access portals between a storage position with all of the capsule receptacles of the capsule rack located within the respective one of the plurality of enclosed volumes and a use position with at least one of the capsule receptacles of the capsule rack located outside the respective one of the plurality of enclosed volumes.

8. The storage device of claim 7, each of the plurality of access portals being located at the same height and arranged linearly.

9. The storage device of claim 7, the plurality of access portals including a first subset and a second subset, the first subset located at a first height, and the second subset located at a second height that is greater than the first height.

10. The storage device of claim 7, the plurality of capsule racks including a first subgroup having capsule receptacles adapted to accommodate personal care capsules of a first size and a second subgroup having capsule receptacles adapted to accommodate personal care capsules of a second size.

11. The storage device of claim 1, the base adapted for selective connection to the container.

12. The storage device of claim 11, the base including a stand adapted for placement on a flat surface, the stand connected to a holder adapted to selectively hold the container.

13. A method of manufacturing a personal care capsule, the method comprising:
preparing a membrane solution including about 0.5-6% calcium lactate;
pouring the membrane solution into a capsule shell mold;
freezing the personal care solution within the capsule shell mold;
removing the personal care solution from the capsule mold as a capsule shell;
bathing the capsule shell in about 1.0-0.5% sodium alginate to create the personal care capsule; and
rinsing the personal care capsule in water.

14. The method of claim 13, further comprising placing the personal care capsule on a capsule rack of a storage device.

15. The method of claim 14, further comprising submerging the personal care capsule in a storage solution located within a container of the storage device.

16. The method of claim 15, the personal care fluid including an active ingredient and the storage solution including the same active ingredient.

17. A personal care capsule, the personal care capsule comprising:
a capsule shell including 0.5-6% calcium lactate, the capsule shell having an interior volume capable of containing a mass of a personal care fluid between 0.5 grams and 5 grams;
the personal care fluid contained within the capsule shell, the personal care fluid including an active ingredient.

18. The personal care capsule of claim 16, the capsule shell including an indicator indicating the active ingredient of the personal care fluid contained within the capsule shell.

19. The personal care capsule of claim 16, the capsule shell being biodegradable.

20. The personal care capsule of claim 16, the capsule shell having a diameter between about 10 and 15 mm.
